(19) United States
(12) Patent Application Publication
(10) Pub. No.: US 2024/0286765 A1
(43) Pub. Date: Aug. 29, 2024

(54) METHOD FOR MAINTENANCE OF AN AIRCRAFT AIR CONDITIONING INSTALLATION, AND AIRCRAFT AIR CONDITIONING SYSTEM

(71) Applicant: Airbus Operations GmbH, HAMBURG (DE)

(72) Inventors: Paul CHEN, HAMBURG (DE); Markus KOENIG, HAMBURG (DE); Joerg BAUMANN, HAMBURG (DE); Michael KAUFHOLD, HAMBURG (DE)

(21) Appl. No.: 18/246,926

(22) PCT Filed: Sep. 23, 2021
(86) PCT No.: PCT/EP2021/076217

§ 371 Date: Aug. 29, 2023

(30) Foreign Application Priority Data
Sep. 28, 2020 (DE) 10 2020 125 202.1

(51) Int. Cl. B64F 5/40; B64D 13/06; B64F 5/30

(52) U.S. Cl. CPC B64F 5/40; B64D 13/06; B64F 5/30; B64D 2013/0618; B64D 2013/0688

(57) ABSTRACT

In a method for maintaining an aircraft air conditioning installation (10), a maintenance control device (100) is connected to a control system (12) of the aircraft air conditioning installation (10). A decontamination activation signal output by the maintenance control device (100) for the activation of a decontamination process is transmitted to the control system (12) of the aircraft air conditioning installation (10). In response to receiving the decontamination activation signal, the control system (12) of the aircraft air conditioning installation (10) controls the operation of the aircraft air conditioning installation (10) such that at least a partial region to be decontaminated of an air distribution system (20, 34, 38) of an aircraft equipped with the aircraft air conditioning installation (10) is heated to a decontamination temperature that is higher than an operating temperature of the partial region of the air distribution system (20, 34, 48) during normal operation of the aircraft air conditioning installation (10).

The invention relates to a method for the maintenance of an aircraft air conditioning installation, and to an aircraft air conditioning system.

On board an aircraft, in particular a passenger aircraft, the occurrence of bad odors is to be avoided to the greatest possible extent. Reducing bad odors is usually possible by reducing the water content in an air distribution system of the aircraft, since dry air is usually perceived as “fresher” than moist air. In addition, it may be necessary to remove undesirable substances, such as bacteria, viruses, or the like, from the aircraft's air distribution system.

It is the object of the invention to provide a method of maintenance for an aircraft air conditioning installation and an aircraft air conditioning system that provide a controlled decontamination of an air distribution system of an aircraft in order to eliminate bad odors and/or undesirable substances.

This object is achieved by a method for maintenance of an aircraft air conditioning installation having the features of patent claim 1 and an aircraft air conditioning system having the features of patent claim 9.

In a method for maintaining an aircraft air conditioning installation, a maintenance control device is connected to a control system of the aircraft air conditioning installation. The maintenance control device is preferably a control device that is temporarily connected to the control system of the aircraft air conditioning installation by a maintenance crew during ground operations of the aircraft. For example, the maintenance control device may be in the form of a portable control device that is brought on board the aircraft by the maintenance crew. Alternatively, however, it is also conceivable to use a control device provided on board the aircraft as a maintenance control device that can be activated by the maintenance crew for example during ground operations of the aircraft.

In the next step, a decontamination activation signal output by the maintenance control device is transmitted to the control system of the aircraft air conditioning installation in order to activate a decontamination process. The control system of the aircraft air conditioning installation can include a plurality of control devices for controlling the operation of the various regions of the aircraft air conditioning installation. For example, the control system of the aircraft air conditioning installation may include a bleed air system control device, a pack control device, and a central zone control device.

The bleed air system control device can be provided to control the operation of a bleed air system of the aircraft air conditioning installation and, in particular, the supply of bleed air from an engine or an auxiliary engine of the aircraft to the aircraft air conditioning installation. The pack control device is preferably arranged to control the operation of an air conditioning pack of the aircraft air conditioning installation. The central zone control device is preferably set up to control the supply of appropriately temperature-controlled conditioned air to the individual air conditioning zones of an aircraft cabin. For example, the central zone control device may be set up to control the operation of air conditioning components, such as valves, blowers, etc., associated with a conditioned air supply system of the aircraft air conditioning installation. All control devices of the control system of the aircraft air conditioning installation are preferably connected to a flight warning system, which is set up to issue an appropriate warning signal in the event of a malfunction of the aircraft air conditioning installation. In addition, a status display may be provided, which may indicate that a decontamination of at least a partial region of an air distribution system of the aircraft is being carried out.

The output of the decontamination activation signal by the maintenance control device may be manually initiated, for example when it is determined during a previous flight or by the maintenance crew during ground operations of the aircraft that a decontamination of at least a partial region of an air distribution system of the aircraft is desired or required to eliminate bad odors and/or undesirable substances. Alternatively, or in addition thereto, however, it is also conceivable for the maintenance control device to automatically output the decontamination activation signal, for example when the maintenance control device receives a signal from a corresponding measuring device or a control device of the aircraft indicating that a decontamination of the air distribution system of the aircraft is desired or required. The partial region of the air distribution system to be decontaminated may also be selected manually by the maintenance crew or automatically in response to the receipt of a corresponding signal that is output to the maintenance control device by a measurement device or control device.

In response to receiving the decontamination activation signal, the control system of the aircraft air conditioning installation controls the operation of the aircraft air conditioning installation such that at least a partial region to be decontaminated of the air distribution system of an aircraft equipped with the aircraft air conditioning installation is heated to a decontamination temperature that is higher than an operating temperature of the partial region of the air distribution system during normal operation of the aircraft air conditioning installation. The heating to the decontamination temperature increases the water vapor partial pressure in the region of the air distribution system exposed to the decontamination temperature, and consequently reduces the water content in this region of the air distribution system. In this way, unpleasant odors can be eliminated. In addition, through the elevated temperatures undesirable substances such as bacteria such as bacteria, viruses, or the like are removed from the air distribution system.

The method according to the invention for maintenance of an aircraft air conditioning installation enables an automatic controlled decontamination of the air distribution system of the aircraft air conditioning installation that is carried out in an efficient manner under the control of the control system of the aircraft air conditioning installation even under difficult ambient conditions, e.g. cold ambient temperatures. The time-intensive use of simulation tools, which allow a manual temperature increase in the air distribution system of the aircraft air conditioning installation by bypassing the sensors and control devices of the aircraft air conditioning installation, can therefore advantageously be dispensed with. In addition, overheating of individual partial regions and/or components of the air distribution system is reliably prevented.

Preferably, in response to receiving the decontamination activation signal, the control system of the aircraft air conditioning installation controls the operation of the aircraft air conditioning installation such that the decontamination temperature is maintained for a defined period of time in the partial region of the air distribution system to be decontaminated. The time period is preferably specified by the decontamination activation signal issued by the maintenance control device and can be adapted to the respective decontamination task. For example, a longer period of time may be provided for maintaining the decontamination temperature if it is determined that a large amount of water is contained in the partial region to be decontaminated of the air distribution system, or if particular bacteria and/or viruses are to be removed in a targeted manner from the partial region to be decontaminated of the air distribution system.

Additionally or alternatively, the control system of the aircraft air conditioning installation may control the operation of the aircraft air conditioning installation to decontaminate at least a partial region of the air distribution system as a function of at least one environmental parameter. For example, an essential environmental parameter that is preferably taken into account in the controlling of the operation of the aircraft air conditioning installation for the decontamination of at least a partial region of the air distribution system is the ambient temperature.

For example, in cold ambient temperatures, it may be difficult to control a compressor of the aircraft air conditioning installation separately so as to heat the compressor to the desired decontamination temperature. However, in cold ambient temperatures, the control system of the aircraft air conditioning installation can respond to these more difficult conditions by, for example, controlling the operation of components upstream of the compressor in such a way that warmer air is already supplied to the compressor and the compressor can thus be heated to the desired decontamination temperature.

To activate decontamination of a bleed air system of the air distribution system, the maintenance control device may output a bleed air system decontamination activation signal to the control system of the aircraft air conditioning installation. In response to receiving the bleed air system decontamination activation signal, the control system of the aircraft air conditioning installation may then control the operation of a bleed air system of the aircraft air conditioning installation in such a way that the bleed air system of the air distribution system is heated to a decontamination temperature that is higher than the operating temperature of the bleed air system during normal operation of the aircraft air conditioning installation. This enables a targeted, controlled decontamination of the bleed air system of the air distribution system. The bleed air system preferably comprises at least one bleed air line connecting an engine and/or an auxiliary engine of the aircraft to an air conditioning pack, in particular to a primary heat exchanger of the air conditioning pack, as well as components connected to this line, such as valves or the like. Further, the bleed air system may include a bleed air temperature sensor for monitoring the temperature of the bleed air supplied to the air conditioning pack.

The decontamination of the bleed air system is preferably controlled by the bleed air system control device of the aircraft air conditioning control system, which device is provided to control the supply of bleed air from an engine or an auxiliary engine of the aircraft to the aircraft air conditioning installation during normal operation of the aircraft air conditioning installation. Accordingly, the bleed air system control device has access to all major components of the bleed air system to be controlled, and can therefore carry out all control interventions necessary to heat the bleed air system to the decontamination temperature.

Preferably, the bleed air system of the air distribution system is heated to a decontamination temperature of about 220 to 240° C., in particular about 230° C. At this temperature, a proper decontamination of the bleed air system is ensured. At the same time, overheating of components installed in the bleed air system of the air distribution system is reliably prevented.

An aircraft air conditioning primary heat exchanger connected to a bleed air line may selectively be included in or excluded from a process for decontaminating the bleed air system. For example, the primary heat exchanger may be included in the process for decontaminating the bleed air system if the primary heat exchanger is also to be decontaminated and/or if air having an increased temperature is to be supplied to a compressor situated downstream of the primary heat exchanger. This may be desirable or necessary for example if the compressor is also to be decontaminated but cannot by itself be heated to the desired decontamination temperature in the presence of cold ambient temperatures. To include or exclude the primary heat exchanger from the process of decontaminating the bleed air system, a bleed air control valve situated upstream of the heat exchanger in the bleed air line can be correspondingly controlled, for example by the bleed air system control device.

To activate decontamination of a compressor region of the air distribution system, the maintenance control device can output a compressor region decontamination activation signal to the control system of the aircraft air conditioning installation. In response to receiving the compressor region decontamination activation signal, the control system of the aircraft air conditioning installation may then control the operation of a compressor of the aircraft air conditioning installation in such a way that the compressor region of the air distribution system is heated to a decontamination temperature that is higher than the operating temperature of the compressor region during normal operation of the aircraft air conditioning installation. This enables a targeted, controlled decontamination of the compressor region of the air distribution system. The compressor region preferably includes a compressor that forms a component of an air conditioning pack of the aircraft air conditioning installation. Further, aircraft air conditioning installation components situated downstream of the compressor may be associated with the compressor region.

The decontamination of the compressor region is preferably controlled by the pack control device of the control system of the aircraft air conditioning installation, which device is provided to control, during normal operation, the operation of the components of the aircraft air conditioning installation provided in the compressor region, and in particular the components of the air conditioning pack of the aircraft air conditioning installation. Accordingly, the pack control device has access to all essential components to be controlled that are installed in the compressor region, and can therefore carry out all control interventions necessary to heat the compressor region to the decontamination temperature.

Preferably, the compressor region of the air distribution system is heated to a decontamination temperature of about 210 to 230° C., in particular about 220° C. At this temperature, a proper decontamination of the compressor region is ensured. At the same time, overheating of components installed in the compressor region of the air distribution system is reliably prevented.

Preferably, the compressor region includes a main heat exchanger of the aircraft air conditioning installation, connected to an outlet of the compressor. Accordingly, the main heat exchanger can advantageously be included in a process for decontaminating the compressor region, and can likewise be decontaminated. Furthermore, other components of the aircraft air conditioning installation situated downstream of the compressor or the main heat exchanger, such as a reheater, a condenser, a turbine, and corresponding lines and valves, may also be associated with the compressor region.

To activate a decontamination of a conditioned air supply system of the air distribution system, the maintenance control device can output a conditioned air supply system decontamination activation signal to the control system of the aircraft air conditioning installation. In response to receiving the conditioned air supply system decontamination activation signal, the control system of the aircraft air conditioning installation may control the operation of an air conditioning pack, connected to the conditioned air supply system of the air distribution system, of the aircraft air conditioning installation, the operation of at least one trim air valve provided in the conditioned air supply system of the air distribution system, and/or the operation of at least one recirculation air blower provided in the conditioned air supply system of the air distribution system, such that the conditioned air supply system of the air distribution system is heated to a decontamination temperature that is higher than the operating temperature of the conditioned air supply system during normal operation of the aircraft air conditioning installation. This enables a targeted, controlled decontamination of the conditioned air supply system of the air distribution system.

Preferably, the conditioned air supply system includes at least one line connecting an air conditioning pack of the aircraft air conditioning installation to an area of an aircraft cabin that is to be air conditioned. Further, the conditioned air supply system may have associated therewith components of the aircraft air conditioning installation that control the flow of conditioned air from an air conditioning pack into an area of the aircraft cabin to be air conditioned, such as corresponding blowers, valves, and the like. A mixing chamber in which conditioned air provided by the air conditioning pack is mixed with recirculation air discharged from the aircraft cabin, at least one recirculation blower, at least one recirculation air line connecting an area of the aircraft cabin to be air conditioned to the mixing chamber, a trim air valve, and a trim air line set up to conduct trim air into the mixing chamber may also be associated with the conditioned air supply system.

The decontamination of the conditioned air supply system is preferably controlled by the pack control device and/or the central zone control device of the control system of the aircraft air conditioning installation. By including the pack control device in a process for decontaminating the conditioned air supply system, the temperature of the air at the outlet of the air conditioning pack can be controlled as desired before entering the conditioned air supply system. In particular, temperature of the air at the air conditioning pack outlet can be increased in order to heat the conditioned air supply system to the desired increased decontamination temperature. The central zone control device is provided, during normal operation of the aircraft air conditioning installation, to control the operation of the components of the aircraft air conditioning installation provided in the conditioned air supply system, in particular valves and blowers, which enable the supply of conditioned air and recirculation air into the mixing chamber of the aircraft air conditioning installation and further into an area of the aircraft cabin to be air conditioned. By appropriately controlling the trim air valve, warm trim air can be supplied as needed to heat the conditioned air supply system to the desired increased decontamination temperature.

Preferably, the conditioned air supply system of the air distribution system is heated to a decontamination temperature of about 60 to 80° C., in particular about 70° C. At this temperature, a proper decontamination of the conditioned air supply system is ensured. At the same time, overheating of components installed in the compressor region of the air distribution system is reliably prevented.

The decontamination temperature in the conditioned air supply system of the air distribution system is preferably determined as a function of an allowable maximum temperature of conditioned air conducted into an avionics region of the aircraft cabin. This prevents overheating of electronic components installed in the avionics region.

An aircraft air conditioning system comprises an aircraft air conditioning installation having a control system. Further, the aircraft air conditioning system includes a maintenance control device that is connectable to the control system of the aircraft air conditioning installation and is set up to output a decontamination activation signal for the activation of a decontamination process and to transmit said signal to the control system of the aircraft air conditioning installation. The control system of the aircraft air conditioning installation is set up to control, in response to receiving the decontamination activation signal, the operation of the aircraft air conditioning installation such that at least a partial region to be decontaminated of an air distribution system of an aircraft equipped with the aircraft air conditioning installation is heated to a decontamination temperature that is higher than an operating temperature of the partial region of the air distribution system during normal operation of the aircraft air conditioning installation.

Preferably, the control system of the aircraft air conditioning installation is set up to control the operation of the aircraft air conditioning installation such that the decontamination temperature is maintained for a defined period of time in the partial region of the air distribution system to be decontaminated. Further, the control system of the aircraft air conditioning installation can be set up to control the operation of the aircraft air conditioning installation to decontaminate at least a partial region of the air distribution system as a function of at least one environmental parameter, in particular as a function of an ambient temperature. In addition, the control system of the aircraft air conditioning installation may be set up to control the operation of the aircraft air conditioning installation to decontaminate at least a partial region of the air distribution system as a function of an operating state of at least one recirculation air blower and/or as a function of an operating state of a trim air system, in particular at least one trim air valve. The goal here is to generate a temperature that is as high as possible also for heating the cabin and the cockpit, in particular in order to eliminate viruses and/or bacteria, without exceeding the permitted temperatures in an avionic air supply line.

The maintenance control device is preferably set up to output a bleed air system decontamination activation signal to the control system of the aircraft air conditioning installation for the activation of a decontamination of a bleed air system of the air distribution system. The control system of the aircraft air conditioning installation can be set up to control, in response to receiving the bleed air system decontamination activation signal, the operation of a bleed air system of the aircraft air conditioning installation such that the bleed air system of the air distribution system is heated to a decontamination temperature that is higher than the operating temperature of the bleed air system during normal operation of the aircraft air conditioning installation.

The control system of the aircraft air conditioning installation may include a bleed air system control device that is set up to control the decontamination of the bleed air system. Further, the control system of the aircraft air conditioning installation may include a central zone control device. The central zone control device may be set up to forward the bleed air system decontamination activation signal output by the maintenance control device to the bleed air system control device. The decontamination temperature in the bleed air system of the air distribution system is preferably about 220 to 240° C., in particular about 230° C. The bleed air system control device can be set up to selectively include a primary heat exchanger, connected to a bleed air line, of the aircraft air conditioning installation in a process for decontaminating the bleed air system or to exclude it from this process. To achieve this, the bleed air system control device may be set up to correspondingly control a bleed air control valve situated upstream of the primary heat exchanger in the bleed air line.

The maintenance control device is preferably further set up to output a compressor region decontamination activation signal to the control system of the can [sic] aircraft air conditioning installation for the activation of a decontamination of a compressor region of the air distribution system. The control system of the aircraft air conditioning installation can be set up to control, in response to receiving the compressor region decontamination activation signal, the operation of a compressor of the aircraft air conditioning installation such that the compressor region of the air distribution system is heated to a decontamination temperature that is higher than the operating temperature of the compressor region during normal operation of the aircraft air conditioning installation.

The control system of the aircraft air conditioning installation may include a pack control device that is set up to control the decontamination of the compressor region. The central zone control device may be set up to forward the compressor region decontamination activation signal output by the maintenance control device to the pack control device. The decontamination temperature in the compressor region of the air distribution system is preferably about 210 to 230° C., in particular about 220° C. The compressor region preferably comprises a main heat exchanger, connected to an outlet of the compressor, of the aircraft air conditioning installation.

The maintenance control device can further be set up to output a conditioned air supply system decontamination activation signal to the control system of the aircraft air conditioning installation for the activation of a decontamination of a conditioned air supply system of the air distribution system. The control system of the aircraft air conditioning installation can be set up to control, in response to receiving the conditioned air supply system decontamination activation signal, the operation of an air conditioning pack, connected to the conditioned air supply system of the air distribution system, of the aircraft air conditioning installation, the operation of at least one trim air valve provided in the conditioned air supply system of the air distribution system, and/or the operation of at least one recirculation air blower provided in the conditioned air supply system of the air distribution system, such that the conditioned air supply system of the air distribution system is heated to a decontamination temperature that is higher than the operating temperature of the conditioned air supply system during normal operation of the aircraft air conditioning installation.

The central zone control device and/or pack control device of the aircraft air conditioning installation can be set up to control the decontamination of the conditioned air supply system. The central zone control device may be set up to forward the conditioned air supply system decontamination activation signal output by the maintenance control device to the pack control device. The decontamination temperature in the compressor region of the air distribution system is preferably about 60 to 80° C., in particular about 70° C. The central zone control device and/or the pack control device is/are preferably set up to determine the decontamination temperature in the conditioned air supply system of the air distribution system as a function of a permissible maximum temperature of conditioned air conducted into an avionics region.

A preferred embodiment of the invention is now explained in greater detail on the basis of the accompanying schematic drawings, of which

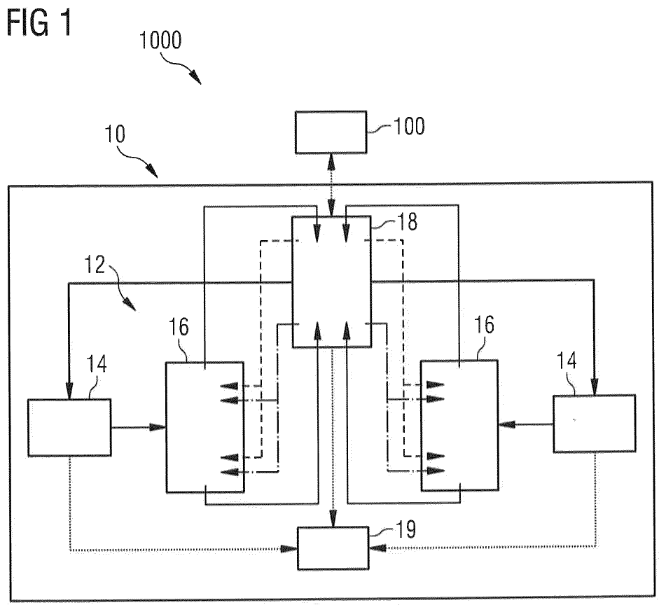

FIG. 1 shows an aircraft air conditioning system with a schematically indicated aircraft air conditioning installation and a maintenance control device, the maintenance control device being connected to a control system of the aircraft air conditioning installation;

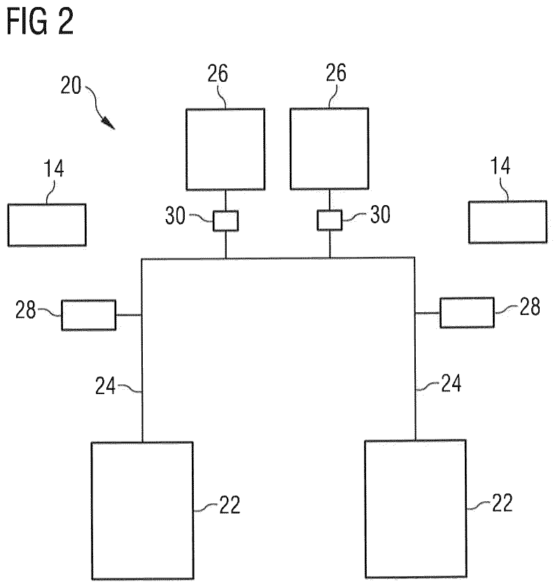

FIG. 2 shows a bleed air system of an air distribution system of the aircraft air conditioning installation;

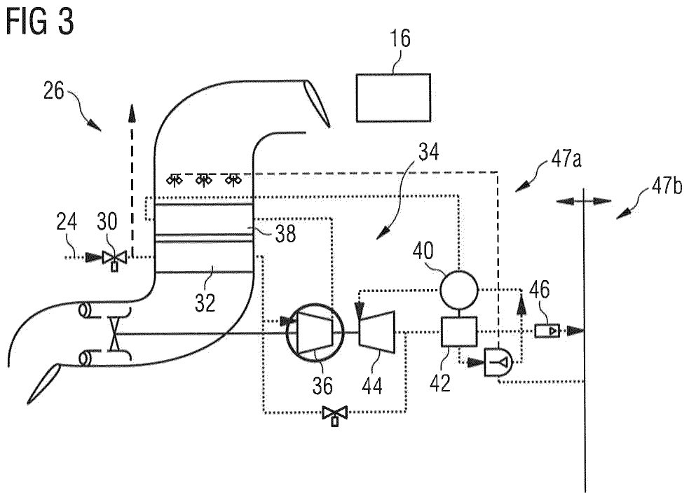

FIG. 3 shows a compressor region of an air distribution system of the aircraft air conditioning installation; and

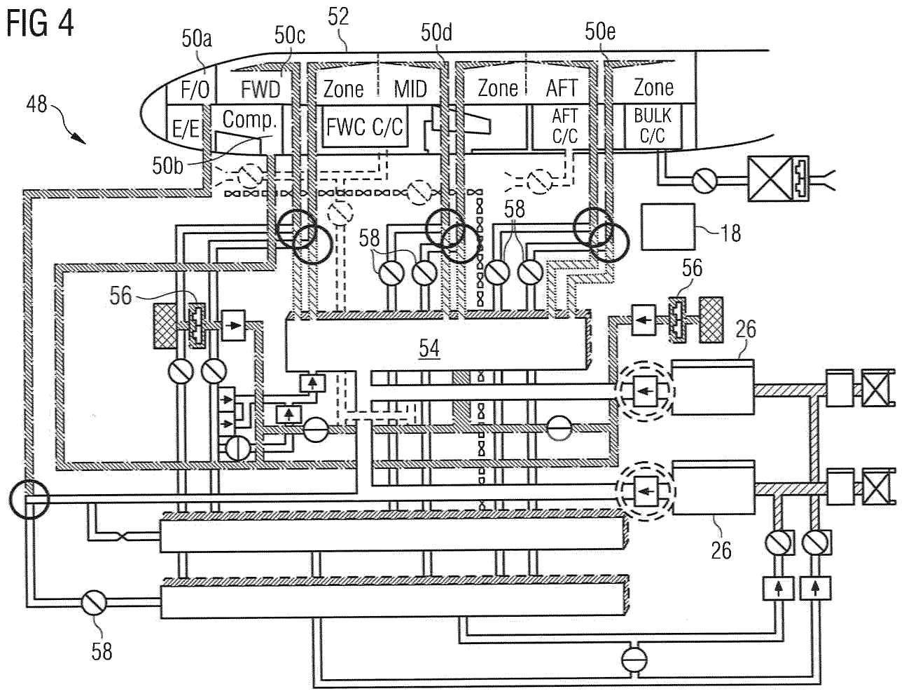

FIG. 4 shows a conditioned air supply system of the aircraft air conditioning installation.

FIG. 1 shows an aircraft air conditioning system 1000 having one of the [sic] aircraft air conditioning installation 10 shown only schematically in FIG. 1 and a maintenance control device 100. In its embodiment shown here, the maintenance control device 100 is designed in the form of a portable control device that is brought aboard the aircraft by a maintenance crew during ground operations of an aircraft equipped with the aircraft air conditioning installation 10 and is temporarily connected to a control system 12 of the aircraft air conditioning installation 10. Alternatively, an internal maintenance interface can also be used.

The control system 12 of the aircraft air conditioning installation 10 includes two bleed air system control devices 14, two pack control devices 16, and a central zone control device 18. All control devices 14, 16, 18 of the control system 12 of the aircraft air conditioning installation 10 are preferably connected to a flight warning system 19, which is set up to issue an appropriate warning signal in the event of a malfunction of the aircraft air conditioning installation 10.

During normal operation of the aircraft air conditioning installation 10, the bleed air system control devices 14 control the operation of a bleed air system 20 of the aircraft air conditioning installation 10, schematically illustrated in FIG. 2, and in particular the supply of bleed air from an engine 22 or an auxiliary engine of the aircraft to the aircraft air conditioning installation 10. The bleed air system 20 includes two bleed air lines 24, each connecting an engine 22 to an air conditioning pack 26. Further, in the bleed air system 20 components are provided that are not shown in FIG. 3, such as bleed air branch lines for tapping bleed air at various locations on the engine 22, as well as valves and the like for controlling the flow of bleed air through the bleed air lines 24.

A bleed air temperature sensor 28 is assigned to each of the bleed air lines 24 for monitoring the temperature of the bleed air supplied to the corresponding air conditioning pack 26. Signals output by the bleed air temperature sensors 28, indicating the temperature of the bleed air flowing through the corresponding bleed air line 24, are transmitted to the corresponding bleed air system control device 14. Further, a bleed air control valve 30 is provided in each of the bleed air lines 24 and is operated under the control of the corresponding pack control device 16 to control the supply of bleed air to an air conditioning pack 26.

During normal operation of the aircraft air conditioning installation 10, each pack control device 16 controls the operation of an air conditioning pack 26, illustrated in more detail in FIG. 3, of the aircraft air conditioning installation 10. Each air conditioning pack 26 includes a primary heat exchanger 32 connected to a bleed air line 24 and a compressor region 34 situated downstream of the primary heat exchanger 32. The compressor region 34 has associated with it a compressor 36, a main heat exchanger 38 connected to an outlet of the compressor 36, and further components arranged downstream of the compressor 36 or the main heat exchanger 38, in particular a reheater 40, a condenser 42, a turbine 44, and corresponding lines and valves. A pack valve 46 is designed in the form of a check valve and prevents conditioned air from flowing back from a pressurized area 47b of the aircraft into an unpressurized area 47a of the aircraft. A conditioned air supply system 48 shown in FIG. 4 is installed in the pressurized area 47b of the aircraft.

The conditioned air supply system 48 includes a plurality of lines that connect the air conditioning packs 26 to corresponding air conditioning zones 50a, 50b, 50c, 50d, 50e of an aircraft cabin 52. Further, the conditioned air supply system 48 has assigned to it components of the aircraft air conditioning installation 10 that control the flow of conditioned air from an air conditioning pack 26 into an air conditioning zone 50a, 50b, 50c, 50d, 50e of the aircraft cabin 52, in particular corresponding blowers, valves, and the like. A mixing chamber 54 in which conditioned air provided by the air conditioning packs 26 is mixed with recirculation air discharged from the aircraft cabin 52, recirculation blowers 56, recirculation air lines, trim air valves 58, and a trim air line used to conduct hot trim air into the mixing chamber 54 may also be assigned to the conditioned air supply system 48.

During normal operation of the aircraft air conditioning installation 10, the central zone control device 18 controls the supply of appropriately tempered conditioned air to the individual air conditioning zones 50a, 50b, 50c, 50d, 50e. In particular, the central zone control device 18 controls the operation of the valves, blowers, etc. provided in the conditioned air supply system 48.

In a method for maintaining the aircraft air conditioning installation 10, the maintenance control device 100 is connected to the control system 12 of the aircraft air conditioning installation 10.

When the maintenance crew determines that a decontamination of at least a partial region of an air distribution system of the aircraft formed by the bleed air system 20, the compressor region 34, and the conditioned air supply system 48 is desired or required in order to eliminate bad odors and/or undesirable substances, the maintenance crew initiates the output of a decontamination activation signal in order to activate a decontamination process. Here the maintenance crew can select which partial region of the air distribution system is to be decontaminated from a selection menu provided by the maintenance control device 100. The decontamination activation signal output by the maintenance control device 100 is transmitted to the control system 12 of the aircraft air conditioning installation 10.

In response to receiving the decontamination activation signal, the control system 12 of the aircraft air conditioning installation 10 controls the operation of the aircraft air conditioning installation 10 such that at least a partial region to be decontaminated of the air distribution system is heated to a decontamination temperature that is higher than an operating temperature of the partial region of the air distribution system during normal operation of the aircraft air conditioning installation 10. This reduces the water content in this area of the air distribution system, thus eliminating unpleasant odors. In addition, through the increased temperatures undesirable substances such as bacteria, viruses, or the like are removed from the air distribution system. In addition, the flight warning system displays a status message on a system display that a decontamination of at least a partial region of the air distribution system of the aircraft is being carried out.

In response to receiving the decontamination activation signal, the control system 12 of the aircraft air conditioning installation 10 controls the operation of the aircraft air conditioning installation 10 such that the decontamination temperature is maintained for a defined period of time in the partial region of the air distribution system to be decontaminated. This time period is preferably specified by the decontamination activation signal issued by the maintenance control device 100 and is adapted to the respective decontamination task. In particular, a longer period of time is provided for maintaining the decontamination temperature if a large amount of water is contained in the partial region to be decontaminated of the air distribution system, or if particular bacteria and/or viruses are to be removed in a targeted manner from the partial region to be decontaminated of the air distribution system.

In addition, the control system 12 of the aircraft air conditioning installation 10 can be set up to control the operation of the aircraft air conditioning installation 10 for the decontamination of at least a partial region of the air distribution system as a function of at least one environmental parameter, in particular as a function of the ambient temperature. In addition, the control system 12 of the aircraft air conditioning installation 10 controls the operation of the aircraft air conditioning installation 10 for the decontamination of at least a partial region of the air distribution system as a function of the operating state of at least one recirculation air blower 56 and/or as a function of the operating state of the trim air system, in particular at least one trim air valve 58, in order to produce as high a temperature as possible for the heating of the cabin 52 and the cockpit without exceeding the allowable temperatures in an avionic air supply line.

When decontamination of the bleed air system 20 of the air distribution system is to be performed, the maintenance crew selects a corresponding menu item “decontamination bleed air system” in the selection menu of the maintenance control device 100, whereupon the maintenance control device 100 outputs a bleed air system decontamination activation signal to the control system 12 of the aircraft air conditioning installation 10. The bleed air system decontamination activation signal is output to the central zone control device 18, which in turn forwards the signal to one of the bleed air system control devices 14 or to both bleed air system control devices 14.

In response to receiving the bleed air system decontamination activation signal, the control system 12 of the aircraft air conditioning installation 10, i.e., one of the bleed air system control devices 14 or both bleed air system control devices 14, then controls the operation of the bleed air system 20 such that the bleed air system 20 is heated to a decontamination temperature that is higher than the operating temperature of the bleed air system 20 during normal operation of the aircraft air conditioning installation 10. This is done for example by supplying bleed air to the bleed air system 20 that has a higher temperature than during normal operation of the bleed air system 20. Preferably, the bleed air region 20 is heated to a decontamination temperature of about 220 to 240° C., preferably about 230° C. during normal operation, on the other hand, the bleed air system 20 is usually operated at a temperature of about 200° C. Monitoring of the temperature in the bleed air system 20 is done using the bleed air temperature sensors 28.

A primary heat exchanger 32 connected to the bleed air line 24 may selectively be included in a process for decontaminating the bleed air system 20 or excluded from this process. For example, the primary heat exchanger 32 may be included in the process for decontaminating the bleed air system 20 if the primary heat exchanger 32 is also to be decontaminated and/or if air having an increased temperature is to be supplied to the compressor 36 situated downstream of the primary heat exchanger 32. In order to include the primary heat exchanger 32 in the process for decontaminating the bleed air system 20, the bleed air control valve 30 situated upstream of the primary heat exchanger 32 in the bleed air line 24 is opened under the control of the pack control device 16. On the other hand, if the primary heat exchanger 32 is to be excluded from the process of decontaminating the bleed air system 20, the bleed air control valve 30 remains closed under the control of the pack control device 16.

When a decontamination of the compressor region 34 of the air distribution system is to be performed, the maintenance crew selects a corresponding menu item “decontamination compressor region” in the selection menu of the maintenance control device 100, whereupon the maintenance control device 100 outputs a compressor region decontamination activation signal to the control system 12 of the aircraft air conditioning installation 10. The compressor region decontamination activation signal is output to the central zone control device 18, which in turn forwards the signal to one of the pack control devices 16 or to both pack control devices 16.

In response to receiving the compressor region decontamination activation signal, the control system 12 of the aircraft air conditioning installation 10, i.e., one of the pack control devices 16 or both pack control devices 16, then controls the operation of the compressor 36 such that the compressor region 34 is heated to a decontamination temperature that is higher than the operating temperature of the compressor region 34 during normal operation of the aircraft air conditioning installation 10. Preferably, the compressor region 34 is heated to a decontamination temperature of about 210 to 230° C., preferably about 220° C. during normal operation of the aircraft air conditioning installation 10, on the other hand, the compressor region 34 is usually operated at a temperature of about 185° C. Heating of the compressor region 34 to the decontamination temperature is accomplished by increasing the operating temperature of the compressor 36, which in turn is accomplished through an appropriate controlling of a ram air inlet cover to a further closed position than during normal operation of the aircraft air conditioning installation 10.

In the case in which under certain environmental conditions, such as cold ambient temperatures, it is not possible to heat the compressor 36 to the desired increased temperature required for decontamination of the compressor region 84, the maintenance crew selects a corresponding menu item “decontamination bleed air system+compressor region” in the selection menu of the maintenance control device 100, whereupon the maintenance control device 100 outputs a bleed air system decontamination activation signal and a compressor region decontamination activation signal to the control system 12 of the aircraft air conditioning installation 10. Further, the bleed air control valve 30 situated upstream of the primary heat exchanger 32 in the bleed air line 24 is opened under the control of the pack control device 16

As a result, air having an increased temperature is supplied to the compressor 36, making it easier to heat the compressor 36 to the desired increased decontamination temperature. Further, when there is such a controlling of the aircraft air conditioning installation 10, the primary heat exchanger 32 situated upstream of the compressor 36 is also included in the decontamination process.

When a decontamination of the conditioned air supply system 48 of the air distribution system is to be performed, the maintenance crew selects a corresponding menu item “decontamination conditioned air supply system” in the selection menu of the maintenance control device 100, whereupon the maintenance control device 100 outputs a conditioned air supply system decontamination activation signal to the control system 12 of the aircraft air conditioning installation 10. The conditioned air supply system decontamination activation signal is output to the central zone control device 18.

In response to receiving the conditioned air supply system decontamination activation signal, the control system 12 of the aircraft air conditioning installation 10, i.e. the central zone control device 18 and/or at least one of the pack control devices 16, controls the operation of at least one air conditioning pack 26 connected to the conditioned air supply system 48, the operation of the trim air valves 58, and/or the operation of at least one recirculation air blower 56 such that the conditioned air supply system 48 is heated to a decontamination temperature that is higher than the operating temperature of the conditioned air supply system 48 during normal operation of the aircraft air conditioning installation 10. The decontamination temperature in the conditioned air supply system 48 is determined as a function of an allowable maximum temperature of conditioned air conducted into an avionics region 50a of the aircraft cabin 52. In particular, the conditioned air supply system 48 is heated to a decontamination temperature of about 60 to 80° C., preferably about 70° C.

The heating of the conditioned air supply system 48 to the decontamination temperature is accomplished by increasing the temperature of the air at the outlet of one or both air conditioning packs 26 before entering the conditioned air supply system 48, under the control of one or both pack control devices 16, supplying warm trim air by opening the trim air valves 58 under the control of the central zone control device 18, and/or corresponding operation of the recirculation blower 56 under the control of the central zone control device 18.

1. A method for maintenance of an aircraft air conditioning installation (10), comprising the steps:
connecting a maintenance control device (100) to a control system (12) of the aircraft air conditioning installation (10); and
transmitting a decontamination activation signal output by the maintenance control device (100) for the activation of a decontamination process to the control system (12) of the aircraft air conditioning installation (10),
wherein, in response to receiving the decontamination activation signal, the control system (12) of the aircraft air conditioning installation (10) controls the operation of the aircraft air conditioning installation (10) such that at least a partial region to be decontaminated of an air distribution system (20, 34, 38) of an aircraft equipped with the aircraft air conditioning installation (10) is heated to a decontamination temperature that is higher than an operating temperature of the partial region of the air distribution system (20, 34, 48) during normal operation of the aircraft air conditioning installation (10).

2. The method according to claim 1,
wherein
the control system (12) of the aircraft air conditioning installation (10) is set up to control the operation of the aircraft air conditioning installation (10) such that the decontamination temperature is maintained for a defined period of time in the partial region to be decontaminated of the air distribution system (20, 34, 38), and/or
the control system (12 of the aircraft air conditioning installation (10) controls the operation of the aircraft air conditioning installation (10) for the decontamination of at least a partial region of the air distribution system (20, 34, 48) as a function of at least one environmental parameter, in particular as a function of an ambient temperature.

3. The method according to claim 1 or 2,
wherein to activate a decontamination of a bleed air system (20) of the air distribution System (20, 34, 48), the maintenance control device (100) outputs a bleed air system decontamination activation signal to the control system (12) of the aircraft air conditioning installation (10), and wherein the control system (12) of the aircraft air conditioning installation (10) controls the operation of the bleed air system (20) of the aircraft air conditioning installation (10) in response to receiving the bleed air system decontamination activation signal such that the bleed air system (20) of the air distribution system (20, 34, 48) is heated to a decontamination temperature that is higher than the operating temperature of the bleed air system (20) during normal operation of the aircraft air conditioning installation (10).

4. The method according to claim 3,
wherein
the decontamination of the bleed air system (20) is controlled by a bleed air system control device (14) of the control system (12) of the aircraft air conditioning installation (10), and/or
the bleed air system decontamination activation signal output by the maintenance control device (100) is forwarded by a central zone control device (18) of the control system (12) of the aircraft air conditioning installation (10) to the bleed air system control device (14) of the control system (12) of the aircraft air conditioning installation (10), and/or
the bleed air system (20) of the air distribution system (20, 34, 48) is heated to a decontamination temperature of about 220 to 240° C., in particular about 230° C., and/or
a primary heat exchanger (32), connected to a bleed air line (24), of the aircraft air conditioning installation (10) is selectively included in a process for decontaminating the bleed air system (24) or is excluded from this process, in particular by a corresponding controlling of a bleed air control valve (30) situated upstream of the primary heat exchanger (32) in the bleed air line (24).

5. The method according to one of claims 1 through 4,
wherein to activate a decontamination of a compressor region (34) of the air distribution system (20, 34, 48), the maintenance control device (100) outputs a compressor region decontamination activation signal to the control system (12) of the aircraft air conditioning installation (10), and wherein the control system (12) of the aircraft air conditioning installation (10) controls the operation of a compressor (36) of the aircraft air conditioning installation (10) in response to receiving the compressor region decontamination activation signal such that the compressor region (34) of the air distribution system (20, 34, 48) is heated to a decontamination temperature that is higher than the operating temperature of the compressor region (34) during normal operation of the aircraft air conditioning installation (10).

6. The method according to claim 5,
wherein
the decontamination of the compressor region (34) is controlled by a pack control device (16) of the control system (12) of the aircraft air conditioning installation (10), and/or
the compressor region decontamination activation signal output by the maintenance control device (100) is forwarded by the central zone control device (18) of the control system (12) of the aircraft air conditioning installation (10) to the pack control device (16) of the control system (12) of the aircraft air conditioning installation (10), and/or
the compressor region (34) of the air distribution system (20, 34, 48) is heated to a decontamination temperature of about 210 to 230° C., in particular about 220° C. and/or
the compressor region (34) includes a main heat exchanger (38) of the aircraft air conditioning installation (10), connected to an outlet of the compressor (36).

7. The method according to one of claims 1 through 6,
wherein to activate a decontamination of a conditioned air supply system (48) of the air distribution system (20, 34, 48), the maintenance control device (100) outputs a conditioned air supply system decontamination activation signal to the control system (12) of the aircraft air conditioning installation (10), and wherein in response to receiving the conditioned air supply system decontamination activation signal, the control system (12) of the aircraft air conditioning installation (10) controls the operation of an air conditioning pack (26) of the aircraft air conditioning installation (10) connected to the conditioned air supply system (48) of the air distribution system (20, 34, 48), the operation of at least one trim air valve (58) situated in the conditioned air supply system (48) of the air distribution system (20, 34, 48), and/or the operation of at least one recirculation air blower (56) situated in the conditioned air supply system (48) of the air distribution system (20, 34, 48) such that the conditioned air supply system (48) of the air distribution system (20, 34, 48) is heated to a decontamination temperature that is higher than the operating temperature of the conditioned air supply system (48) during normal operation of the aircraft air conditioning installation (10).

8. The method according to claim 7,
wherein
the decontamination of the conditioned air supply system (48) is controlled by the pack control device (16) and/or the central zone control device (18) of the control system (12) of the aircraft air conditioning installation (10), and/or
the conditioned air supply system decontamination activation signal output by the maintenance control device (100) is forwarded by the central zone control device (18) of the control system (12) of the aircraft air conditioning installation (10) to the pack control device (16) of the control system (12) of the aircraft air conditioning installation (10), and/or
the conditioned air supply system (48) of the air distribution system (20, 34, 48) is heated to a decontamination temperature of about 60 to 80° C., in particular about 70° C., and/or
the decontamination temperature in the conditioned air supply system (48) of the air distribution system (20, 34, 48) is determined as a function of an allowable maximum temperature of conditioned air conducted into an avionics region (50a) of the aircraft cabin.

9. An aircraft air conditioning system (1000) comprising:
an aircraft air conditioning installation (10) having a control system (12) and
a maintenance control device (100) that is connectable to the control system (12) of the aircraft air conditioning installation (10) and is set up to output a decontamination activation signal for the activation of a decontamination process and to transmit said signal to the control system (12) of the aircraft air conditioning installation (10), wherein the control system (12) of the aircraft air conditioning installation (10) is set up to control, in response to receiving the decontamination activation signal, the operation of the aircraft air conditioning installation (10) such that at least a partial region to be decontaminated of an air distribution system (20, 34, 38) of an aircraft equipped with the aircraft air conditioning installation (10) is heated to a decontamination temperature that is higher than an operating temperature of the partial region of the air distribution system (20, 34, 48) during normal operation of the aircraft air conditioning installation (10).

10. The aircraft air conditioning system according to claim 9,
wherein
the control system (12) of the aircraft air conditioning installation (10) is set up to control the operation of the aircraft air conditioning installation (10) such that the decontamination temperature is maintained for a defined period of time in the partial region to be decontaminated of the air distribution system (20, 34, 38), and/or
the control system (12) of the aircraft air conditioning installation (10) is set up to control the operation of the aircraft air conditioning installation (10) for the decontamination of at least a partial region of the air distribution system (20, 34, 48) as a function of at least one environmental parameter, in particular as a function of an ambient temperature.

11. The aircraft air conditioning system according to claim 9 or 10,
wherein to activate a decontamination of a bleed air system (20) of the air distribution system (20, 34, 48), the maintenance control device (100) outputs a bleed air system decontamination activation signal to the control system (12) of the aircraft air conditioning installation (10), and wherein the control system (12) of the aircraft air conditioning installation (10) is set up to control, in response to receiving the bleed air system decontamination activation signal, the operation of the bleed air system (20) of the aircraft air conditioning installation (10) such that the bleed air system (20) of the air distribution system (20, 34, 48) is heated to a decontamination temperature that is higher than the operating temperature of the bleed air system (20) during normal operation of the aircraft air conditioning installation (10).

12. The aircraft air conditioning system according to one of claims 9 through 11, wherein to activate a decontamination of a compressor region (34) of the air distribution system (20, 34, 48), the maintenance control device (100) is set up to output a compressor region decontamination activation signal to the control system (12) of the aircraft air conditioning installation (10), and wherein the control system (12) of the aircraft air conditioning installation (10) is set up to control, in response to receiving the compressor region decontamination activation signal, the operation of a compressor (36) of the aircraft air conditioning installation (10) such that the compressor region (34) of the air distribution system (20, 34, 48) is heated to a decontamination temperature that is higher than the operating temperature of the compressor region (34) during normal operation of the aircraft air conditioning installation (10).

13. The aircraft air conditioning system according to one of claims 9 through 12, wherein to activate a decontamination of a conditioned air supply system (48) of the air distribution system (20, 34, 48), the maintenance control device (100) is set up to output a conditioned air supply system decontamination activation signal to the control system (12) of the aircraft air conditioning installation (10), and wherein the control system (12) of the aircraft air conditioning installation (10) is set up to control, in response to receiving the conditioned air supply system decontamination activation signal, the operation of an air conditioning pack (26) of the aircraft air conditioning installation (10) to the conditioned air supply system (48) of the air distribution system (20, 34, 48), the operation of at least one trim air valve (58) situated in the conditioned air supply system (48) of the air distribution system (20, 34, 48), and/or the operation of at least one recirculation air blower (56) situated in the conditioned air supply system (48) of the air distribution system (20, 34, 48) such that the conditioned air supply system (48) of the air distribution system (20, 34, 48) is heated to a decontamination temperature that is higher than the operating temperature of the conditioned air supply system (48) during normal operation of the aircraft air conditioning installation (10)
